(19) United States
(12) Patent Application Publication
(10) Pub. No.: US 2026/0294336 A1
(43) Pub. Date: Oct. 1, 2026

(54) SYSTEM AND METHOD TO DETERMINE AND REVIEW THERAPY

(71) Applicants: MEDTRONIC, INC., Minneapolis, MN (US); NUTRINO HEALTH LTD., Tel-Aviv-Jaffa (IL)

(72) Inventors: Daniel D. Anderson, Raanana (IL); Elizabeth A. Fehrmann, Falcon Heights, MN (US); Steven M. Goetz, North Oaks, MN (US); Michelle A. Case, Blaine, MN (US); Pavel Mostov, Netanya (IL); Alexa Singer, Berlin (BE); Caleb C. Zarns, Chicago, IL (US); Mohammad Amin Nourmohammadi, New Brighton, MN (US); Rene A. Molina, Maple Grove, MN (US)

(21) Appl. No.: 19/479,464

(22) PCT Filed: Apr. 24, 2024
(86) PCT No.: PCT/US2024/026015

§ 371 Date: Oct. 28, 2025

Related U.S. Application Data
(60) Provisional application No. 63/462,776, filed on Apr. 28, 2023.

(51) Int. Cl. A61B 5/00; A61B 5/11

(52) U.S. Cl. CPC A61B 5/4836; A61B 5/1101; A61B 5/4064; A61B 5/742

(57) ABSTRACT

Disclosed is a system and method to analyze information from a subject. The information may include local field potential signals from the subject. The signals may be recorded over time. The signals may then be analyzed and evaluated relative to the subject and/or other information regarding the subject.

### CROSS-REFERENCE TO RELATED APPLICATIONS

This application claims the benefit of U.S. Provisional Application No. 63/462,776, filed on Apr. 28, 2023. The entire disclosure of the above application is incorporated herein by reference.

### FIELD

The present disclosure relates to a method and system to evaluate sensed signals from a subject over time, particularly related to neurological signals.

### BACKGROUND

This section provides background information related to the present disclosure which is not necessarily prior art.

A system may sense a signal in a human patient. The signal may be referred to as local field potential (LFP) signal which is the electric potential recorded in the extracellular space in brain tissue. The signal may be sensed in a selected portion of the patient, such as at a probe positioned within the brain of the patient. The probe may be temporarily or chronically positioned. The probe may sense the LFP signal over time as a plurality of LFP signals. The LFP signals may be viewed on a timeline.

### SUMMARY

This section provides a general summary of the disclosure, and is not a comprehensive disclosure of its full scope or all of its features.

An implantable device may be configured to measure activity, such as a local field of potential (LFP) and/or transmit the LFP signals of a subject. The subject may be a human patient. The subject, however, may also be in an inanimate subject or a nonhuman patient that generates electrical signals that may be sensed by an implant. The implant may be temporarily or chronically implanted in the subject.

A system may be used to analyze changes in one or more treatment parameter(s) relative to or related to the subject's or patient's LFP. In various embodiments, more than one LFP signal may be sensed and may correspond to spectral features of an LFP signal such as a band passed LFP signals or a mathematical operation of multiple band passed LFP signals. Treatment parameters may include changes in various parameters such as stimulation frequency, stimulation amplitude, or other selected parameters. A change in parameters may be used to identify epochs in an LFP timeline which is a portion in a timeline sequence of an LFP sequence for comparison to other timeline sequences. The change in one or more parameters may be used to identify the end and/or beginning of a timeline sequence as one or more epochs. Sequences may also be referred to as epochs to allow for a comparison of related or different conditions of the subject based upon the same or different parameters applied to the subject. While epoch detection as disclosed herein may relate primarily with LFP signals, one skilled in the art will understand that the disclosure is understood to not be limited to solely LFP signals. Signals may be from other sources (e.g., wearables) to which the epoch determinations can and/or will be applied. Signals for epoch applicability may include “sensed signals” and “signal timelines” as LFP signals are only one example of a (type of) sensed signal.

The system may automatically identify one or more epochs that relate to the subject. One or more epochs may relate to a difference in treatment parameters and/or identified characteristics of the subject. The one or more epochs may be correlated and/or displayed relative to one another for evaluation by a user. Herein, reference to epoch in the singular is intended to refer to one or more, unless specifically indicated otherwise.

According to various embodiments, a system to determine an outcome metric of a subject undergoing a therapy that may include a processor module is disclosed. The processor module may be configured to at least receive signal data regarding a signal from the subject with one or more corresponding signal time stamps and at least receive data regarding one or more parameters with one or more corresponding parameter time stamps. The processor module may further execute instructions to identify one or more changes in the parameters based on one or more change rules (which may be determined and defined in a system and used in the instructions), determine one or more epochs based on the identified one or more changes and the parameter time stamps that correspond to the identified one or more changes, and output at least one first outcome metric for the determined epochs, wherein the first outcome metric comprises associating the signal data within at least one determined epoch.

According to various embodiments, a method for determining an outcome metric of a subject undergoing a therapy is disclosed. The method may include receiving signal data regarding a signal from the subject with one or more corresponding signal time stamps. The method may further include receiving data regarding one or more parameters with one or more corresponding parameter time stamps. The method may further include identifying one or more changes in the parameters based on one or more change rules. The method may further include determining one or more epochs based on the identified one or more changes and the parameter time stamps that correspond to the identified one or more changes. The method may further include outputting at least one first outcome metric for the determined epochs, wherein the first outcome metric comprises associating the signal data within at least one determined epoch.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic view illustrating an exemplary deep brain stimulation (DBS) system, according to various embodiments;

FIG. 2 is a graph of a local field potential (LFP) trace over time;

FIG. 3 is an exemplary graph of various parameters over time;

FIG. 4 is a parameter listing tree, according to various embodiments;

FIG. 5 is a rule change table or look up table, according to various embodiments;

FIG. 6 is a flow chart of a process for evaluating or determining an epoch, according to various embodiments;

FIG. 7 is a schematic illustration of an epoch determination, according to various embodiments;

FIG. 8 is an exemplary display of a right hemisphere epoch determination, according to various embodiments;

FIG. 9 is an exemplary display of a left hemisphere epoch determination, according to various embodiments; and

FIG. 10 is an exemplary display of a graphical user interface.

Corresponding reference numerals indicate corresponding parts throughout the several views of the drawings.

### DETAILED DESCRIPTION

Example embodiments will now be described more fully with reference to the accompanying drawings.

This disclosure is directed towards a system and method for analyzing signals from a subject that may be useful in treating movement disorder symptoms. In one particular example, the disclosure is directed towards treating Parkinson's Disease (PD). In various embodiments, a treatment may include a combination of one or more treatment options such as electrical stimulation, medication, physical motion or placement, or other selected treatment options. In various embodiments, the treatment may include only one selected treatment option.

The systems and methods of this disclosure use sensed brain activity. The brain activity may relate to and/or be useful for determining whether one or more disease state biomarkers are present. Brain activity may be recorded, for example, in the form of a local field potential (LFP). The LFP may be analyzed alone and/or in relation to other sensed or recorded features such as electroencephalogram (EEG) or electrocorticogram (ECoG) signals sensed by an implantable or external medical device. Entrainment generally refers to the process of using stimuli to affect brain activity, e.g., oscillations within a frequency band in the brain. Gamma frequency band oscillations, e.g., ordinarily between about 35 Hertz (Hz) and about 120 Hz or more, in the central nervous system (CNS), recorded using LFP, for example, are associated with normal information processing in movement and sensory structures. Beta frequency band oscillations between about 8 Hz and about 35 Hz, have been associated with dysfunctions of CNS circuits that control behavioral movements and cognitive states. Higher frequency stimulation, e.g., about 130 Hz, of subcortical brain areas involved with movement, e.g., subthalamic nucleus, globus pallidus internus, and ventralis intermedius nucleus of the thalamus, may reduce behaviors associated with essential tremor and Parkinson's disease such as rigidity, bradykinesia, and tremor.

In general, PD patients have phasic changes in their levels of symptom relief and side effects as medication is absorbed into the blood stream, and then is eliminated. For example, a PD patient may show signs of dystonia when not on medication or receiving stimulation. As the treatment (either medication or stimulation) reaches the therapeutic window, the dystonia symptoms may subside.

Brain signals include several biomarkers that may be used to indicate when adjustments to patient treatment may be beneficial to keep a patient within the therapeutic window. Brain signals may be collected from, for example, the patient's motor cortex, zona incerta (Zi), subthalmic nucleus (STN), basal ganglia, cerebellum, pedunculopontine nucleus, red nucleus, or lateral globus pallidus. The signals from the motor cortex may be collected from the primary motor cortex (M1), the premotor cortex, the supplementary motor area (SMA), the posterior parietal cortex, or the primary somatosensory cortex. One or more biomarkers may be found in the signals collected from each region of the brain.

For example, measured LFP signals from the patient's motor cortex and STN may be used as a biomarker and/or may be analyzed as discussed herein for evaluating, such as comparing, different time frames or epochs of a patient. The LFP and/or related biomarker may be monitored by an implantable medical device (IMD) or external programmer or controller. The biomarkers may be used to assess the patient's current disease state. The biomarker may also be used to serve as an indicator of therapy effectiveness in a device or system.

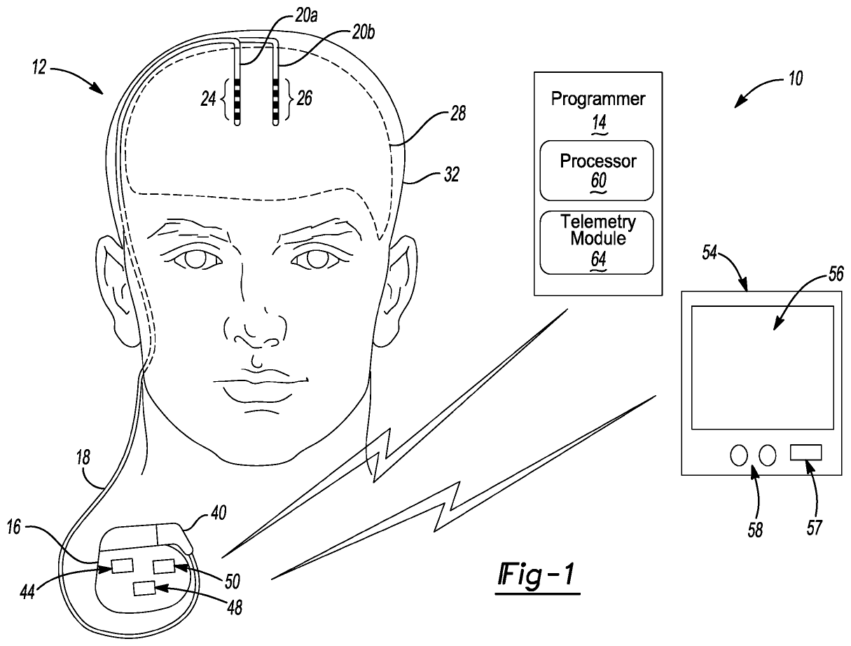

With initial reference to FIG. 1, a schematic or diagram illustrating an example deep brain stimulation (DBS) system that may be used to implement the techniques of this disclosure. In FIG. 1, a therapy and/or recording/measuring system 10 is illustrated. The system 10 may include any appropriate system such as the SenSight® directional deep brain stimulation (DBS) lead system the Percept® PC DBS system and/or related software, with and/or the Brainsense® software that may be used to operate and control a medical device, all sold by Medtronic, Inc. These systems may sense and/or transmit a signal regarding the sensed LFP in a subject. In various embodiments, systems may include features such as those discloses in U.S. Pat. No. 10,016,606, incorporated herein by reference.

The system 10 may deliver electrical stimulation therapy to a patient to assist in treating a patient's condition, such as a movement disorder or a neurodegenerative impairment of a patient 12. The system 10 may also be used to measure and/or record signals from the patient 12, such as LFP signals. Patient 12 may be a human patient. In some cases, however, therapy system 10 may be applied to other mammalian or non-mammalian non-human patients. Further, the system 10 may measure signals in non-living systems, such as electrical signal systems. While movement disorders and neurodegenerative impairment are primarily referred to in this disclosure, in other examples, therapy system 10 may provide therapy to manage symptoms of other patient conditions, such as, but not limited to, seizure disorders or psychological disorders.

A movement disorder or other neurodegenerative impairment may include symptoms such as, for example, muscle control impairment, motion impairment or other movement problems, such as rigidity, bradykinesia, rhythmic hyperkinesia, non-rhythmic hyperkinesia, and akinesia. In some cases, the movement disorder may be a symptom of PD. The movement disorder may be attributable to other patient conditions. Although PD may be referred to here, therapy systems and methods described herein may also be useful for controlling symptoms of other conditions, such as other movement disorders or neurodegenerative impairment.

In the example of FIG. 1, therapy system 10 includes a device programmer 14, subject device 16, a lead or connector 18, and one or more leads 20a and 20b. The subject device 16 may be, such as referred to herein, an implantable medical device (IMD), however one skilled in the art will understand that the device need not be implanted. Each lead 20a, 20b may include one or more electrodes 24, 26. Each electrode 24, 26 may include one or more electrode contacts. It is understood by one skilled in the art, however, that the number of leads 20a, 20b may be greater than two or less than two. Further, the number of electrodes or contacts for each lead may be any appropriate number. The electrode contacts may be used to provide stimulation or record a signal, such as a LFP. In various embodiments, the leads 20a, 20b may be separate leads, or bifurcated segments on a single lead.

In the example shown in FIG. 1, electrodes 24, 26 of leads 20a, 20b may be positioned to sense LFPs and/or deliver electrical stimulation to a tissue site within a brain 28 within a skull 32 of the patient 12, such as a deep brain site under the dura mater of the brain 28 of the patient 12. In various embodiments, the electrodes 24, 26 may be positioned in various regions of the brain 28, such as a subthalamic nucleus (STN), a globus pallidus internus (GPi), a motor cortex, or a thalamus. The placement of the electrodes 24, 26 in these positions may be effective for recording and/or treatment to manage movement disorders, such as Parkinson's Disease or essential tremor.

IMD 16 may include a therapy module that includes a stimulation generator that generates and delivers electrical stimulation therapy to the patient 12 via at least a subset of electrodes 24, 26 of leads 20a and 20b. The subset of electrodes 24, 26 may have a selected and/or changeable polarity and a therapy parameter may be altered thereat, such as frequency and/or changing polarity over time. Further, the stimulation therapy may be altered over time by changing which of the subset of electrodes 24, 26 are combined to provide stimulation.

Using generally known techniques, a subset of electrodes 24, 26, may be used to deliver electrical stimulation to the patient 12 in order to re-establish, or re-induce, gamma frequency band activity within the brain 28. In some examples, beta frequency band activity in the brain may be decreased and gamma frequency band activity in the brain may be increased by delivering electrical stimulation to a portion of the brain at a predetermined ratio between the detected activity in the gamma band and the frequency of stimulation. In one example, the frequency of the electrical stimulation delivered to the portion of the brain may be at a constant frequency at some predetermined ratio between the detected activity in the gamma band and the frequency of electrical stimulation. For example, electrodes 24, 26 may be used to deliver electrical stimulation to patient 12 at a selected frequency and/or provided in a biphasic manner. For example, stimulation may be provided at a particular frequency at a voltage that alternates between +2V and −2V. In another example, the frequency of the electrical stimulation delivered to the portion of the brain may be applied in a sweeping manner. For example, the frequency of the electrical stimulation may be swept through a range of frequency values. In a frequency sweep, the frequency of the electrical stimulation may begin at one value and then may be varied, e.g., increased or decreased, from a first frequency to a second frequency.

The IMD 16 may be connected to the lead 18 at a connection or connector 40. The connector 40 allows the lead 18 to be connected, such as electrically connected, to the IMD 16. The IMD 16 may further include various components or portions such as a battery 44, a processor module 48, and/or a telemetry module 50. The battery 44 may provide power to the IMD 16 such as for stimulating the subject 12 and/or transmitting data. The processor 48 may process various instructions, such as stimulation or sensing instructions. Further, the telemetry module 50 may allow for communication, such as wired or wireless, with the programmer 14 and/or a clinician module or system 54. The telemetry module 50 may be any appropriate module, such as those generally known in the art.

The clinician system 54 may include various portions such as a screen or display 56. The display 56 may be a touch screen display and allow for a clinician or user to input and/or receive and/or review various information. Further, the clinician system 54 may include one or more hard and/or selectable buttons 58. Buttons 58 may allow the user to input or interact with data displayed on the display 56.

The programmer 14 and may be used to program and/or also receive information from of the IMD 16. The programmer 14 may, therefore, include a processor 60 that is able to execute instructions. A memory may also be included with the processor 60 and/or accessible by the processor 60. The programmer 14 may further include a telemetry module 64. Thus, the programmer 14 may communicate with the IMD 16, such as via the telemetry module 50 of the IMD 16. The programmer 14 may send instructions for providing therapy to the subject 12 and/or receiving information from the IMD 16.

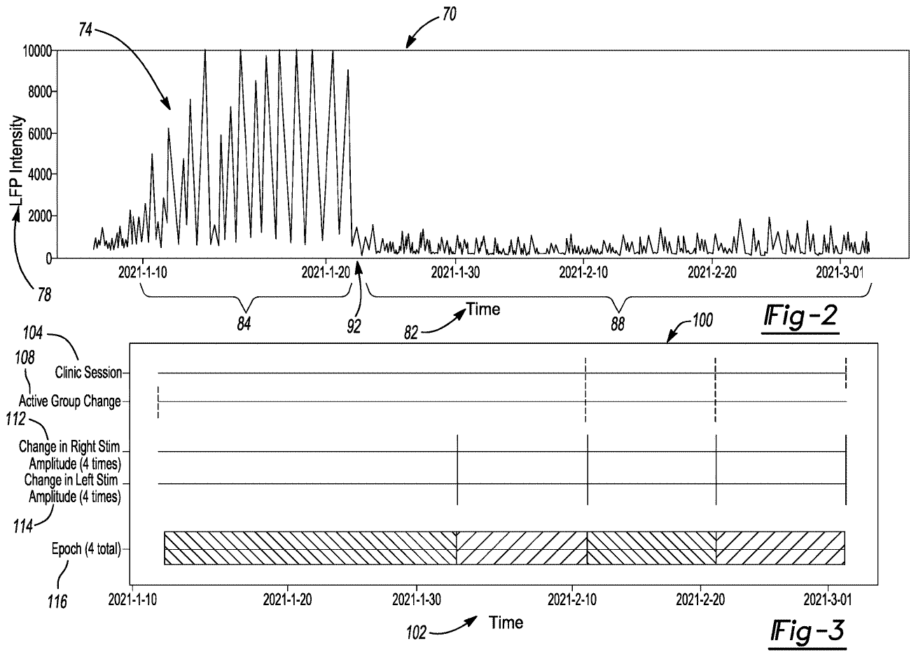

Turning reference to FIG. 2, the system 10, including the electrodes 20a, 20b may be used to sense or record activity in the subject 12. For example, as discussed above, LFP signals may be sensed and measured in the subject 12. FIG. 2 illustrates a graph 70 of LFP signals over time. In the graph 70 an LFP signal trace or line 74 is of a signal that has varying intensity as illustrated on the Y axis 78. The LFP signals may be measured over time as illustrated on the X axis 82. The line 74 of the LFP intensities may illustrate a varying intensity over time, as illustrated in the graph 70. The difference of the intensity of the graph 74 may be viewed by a user.

The user may view the graph 70 on the user system 54. The user, however, may only be able to view a portion time 82 of the graph 74 at any one time on the display 56 of the user system 54. Further, the user may not be able to quickly interpret the graph 74 of the LFP intensity. As illustrated in FIG. 2, the graph 74 of the LFP intensity may include sharp changes in a short period of time and viewing and understanding a change over a long period of time (e.g., greater than a selected value of hours, days, weeks, months, etc.) may be difficult. Further, the graph 70 only illustrates the LFP trace 74 as intensity relative to time. Thus, a user may not be able to understand other factors that occur at a selected one or more changes of the LFP intensity graph 74.

Nevertheless, as discussed further herein, the differentiation of the graph 74 may be evaluated and/or understood relative to other features of the subject 12, the system or system components, or combinations thereof. For example, as illustrated in FIG. 2, a first portion of the graph 74 may have a high average and/or greater peak intensity 84 and a second portion of the trace 74 may have lower average and/or lower peak intensity portion 88. This may also be illustrated over time. An inflection or differentiation time point 92 may be used to evaluate a difference in a treatment to the subject 12 based upon the difference in the LFP intensity as illustrated in the graph 74. However, a significant difference in the LFP intensity over time may not be readily viewable in the graph 70, but may be evaluated relative to other parameters, such as a change in therapy intensity, frequency, or the like.

Turning reference to FIG. 3, a graph or delineation 100 is illustrated. The graph 100 may include a time axis 102 that is identical to the time axis 82 of the LFP 82. The time axis 102 may include a delineation of time. The time axis 102 indicates that the data is collected over time and may allow a timestamp of various data collected regarding one or more parameters of therapy to the subject 12 and/or recording of data from the subject 12. The LFP trace 74 may be traced relative to the time axis 82 and various portions thereof may also be time stamped, as noted above.

As illustrated in the graph 100, various parameters may also be tracked or recorded over time relative to the time axis 102 and/or include time stamps. For example, a clinical session may be identified in line 104. An identification of an active group change may be identified in line 108. The active group change may include which one or more of the electrodes 20a, 20b and/or electrode contacts 24, 26 are activated. Identification of a change may include information regarding what a specific change was, including the active electrodes or electrode contacts. According to various embodiments, particularly if there is more than one electrode (e.g., a right and left brain hemisphere electrode), a stimulation change may be identified regarding a change in a right hemisphere stimulation amplitude in line 112 or a change in a left hemisphere stimulation amplitude in line 114. It is understood that only one electrode may be positioned, but for illustration in the graph 100 both a left and right hemisphere stimulation change may be recorded. Any one of the parameters may be identified over time and/or have a timestamp when the change or a certain parameter is started, ended, or changed.

Using the information, such as that included in the graph 100, may allow for identification of various different time segments, also referred to as periods or portions, which may also be referred to as epochs. An epoch may also be graphed in the graph 100 at line 116. As illustrated in the graph 100 in FIG. 3, four epochs, as an example, may be identified based upon various changes in parameters, including the change in a stimulation amplitude, active group, or any other appropriate parameters. It is understood by one skilled in the art that any appropriate number of epochs may be identified and/or graphed. Graph 100 illustrates only a selected number of parameters, but one skilled in the art understands that any appropriate parameters may be tracked over time. As discussed further herein, according to various embodiments, one or more parameters may be tracked over time and may be used to identify an epoch. The epoch may include time portions that are identified by time stamps or time changes or portions. These time stamps may be related to the LFP trace 74 to evaluate the LFP trace over a selected time or at different timestamps, as discussed further herein.

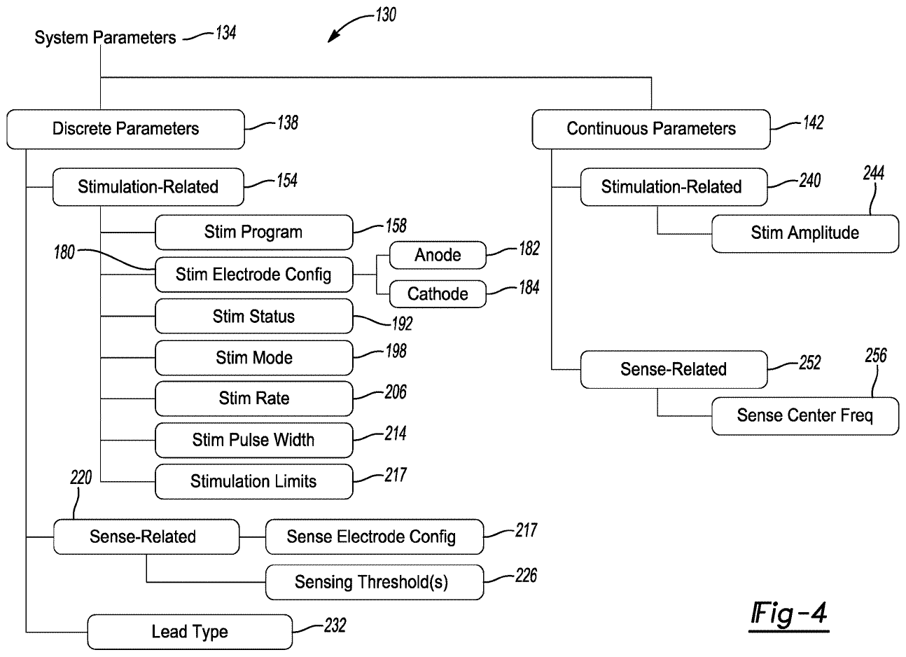

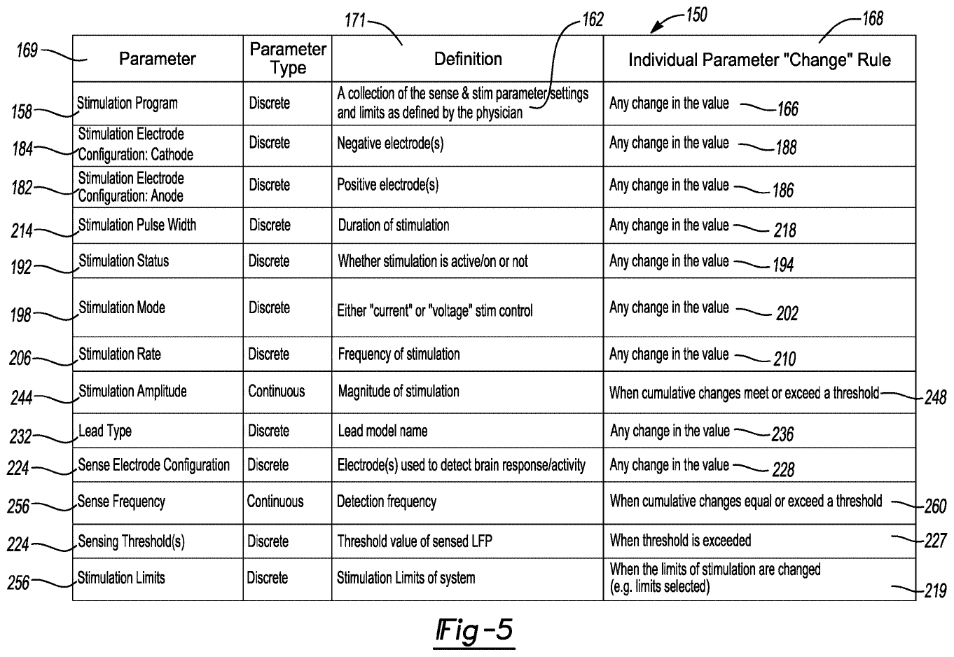

Turning reference to FIG. 4 and FIG. 5, a plurality of parameters may be included in a system, such as the system 10. FIG. 4 illustrates system parameters that may be changed and/or measured. The parameters may be illustrated as a system parameter tree 130 that may include a plurality of system parameters 134 but is only for illustrative purposes to illustrate that various parameters may be classified or grouped together. For example, the system parameters 134 may be divided into discrete parameters 138 or continuous parameters 142. One skilled in the art will understand, however, that this is exemplary and is included here for illustration and discussion of various parameters. However, any parameter may be understood and/or measured to be discrete, continuous, or both.

FIG. 5 is a rule change table or look up table that includes the parameters listed in FIG. 4 along with a brief description or definition and the change rule for each parameter. The “change rule” may refer to how much of a “difference” or how much “variation”, as discussed herein, in a given parameter is considered significant enough to constitute a “change” in the parameter that may be used to identify or define an epoch or epoch sequence. A “difference” in one or more of the parameters may be discrete in nature and may include a type change, a status change, a step change, or any other difference in the parameter deemed relevant. A variation in one or more of the parameters may be continuous in nature and may include an amount and/or proportion change, a cumulative measure or computation crossing some threshold, or any other variation in the parameter deemed relevant.

The system parameters may include parameters that are recorded and/or may be determined relative to the subject 12. The parameters may be selected and/or be default values for the system. The parameters may also or alternatively be configurable and/or adjustable to tailor therapy to patient. These parameters may be stored by the system for use of the system to provide therapy. The parameters illustrated in FIGS. 4 and 5 are exemplary possible parameters that may be selected. Parameters for evaluation and/or determination of an epoch may include one or more of the parameters 130 and/or other parameters. Further, change rules may include alternative or additional rules.

The parameters may include stimulation provided to the subject 12 and/or recording of signals from the subject 12, such as the LFP signals. Regardless, each of the system parameters 134 may include various types of parameters such as one or more discrete parameters 138 and one or more continuous parameters 142. The discrete parameters 138 may include parameters that a change at discrete times and/or by discrete values. The discrete parameters may, therefore, be defined at a significant time, such as when a parameter is changed that is a discrete parameter 138. Continuous parameters 142 may include a value or amount that changes continuously and/or is tracked over time. A continuous parameter may, therefore, be used to define in a parameter change when a selected threshold is reached. With reference to FIG. 5, a rule chart 150 is illustrated. The rule chart 150 may include various definitions that may be used to identify or select when a change happens that may define a new epoch. For example, when a change is significant enough to identify an ending of a previous epoch and/or a beginning of a new epoch. The rule chart 150 may include various rules for various parameters, such as those illustrated in the parameters 134. Therefore, the system parameters 134 may be evaluated relative to a rule as illustrated in the rule chart 152 to allow for a definition or identification of a new epoch or ending an epoch in a data set or relative to the LFP trace 74 for analysis and/or or illustration.

As discussed further herein, the system parameters 134 illustrated in the system parameter tree 130 may have a rule related thereto as illustrated in rule chart 150. For example, the discrete parameters 138 may include stimulation related parameters 154. The stimulation related parameters may include any parameter that relates to a stimulation of the subject, such as through the lead 20a, 20b. Discussion herein of any one specific lead, such as the lead 20a, will be understood to relate to any appropriate lead and/or number of leads, unless specifically indicated otherwise. Therefore, the stimulation related parameters may be discrete parameters such as those discussed further herein.

A stimulation program parameter 158 may be a discrete parameter that is stimulation related. As illustrated in the rule chart 150 the stimulation program parameter 158 may be a discrete parameter that includes a parameter definition 162 of a collection of sense and/or stimulation parameters that may be selected by a user, such as a physician. The stimulation program may include the stimulation parameters, including values and/or changes therefore, such as an amount of stimulation, a frequency of stimulation, a time or amount of a change of stimulation, or the like and/or combinations thereof. The stimulation program 158 may be used to identify an end of a previous epoch or a beginning of a new epoch with any change in value as identified in the change rule 166. Change rules may be identified in a change rule column 168. The change rule chart 150 may include the parameter identification 169 and related information, such as the definition 171 and the change rule 168 for each parameter. Therefore, a processor system, such as the processor 60 of the programmer 14 and/or processor 57 of the user system 54 may search or determine a stimulation parameter change and may identify that any change in value relates to the rule 166 to determine that a change or determination of an epoch must be made. The rule change table 150 may be a look up table that is accessed by the processor, such as the processor 57, in executing instructions of a selected process as discussed herein. In other words, each of the rules have parameters regarding a change, such as the rule for determining a sensing frequency change is that the difference between frequencies is greater than or equal to a selected threshold (e.g., 5 Hz that is a definable parameter for that rule). One skilled in the art will understand, however, that the rules may also be understood to be boundaries conditions for both how changes in parameters are identified (e.g., greater than or equal to a threshold) as well as each rule's parameters (e.g., the threshold is set to 5 Hz).

The stimulation program may be tracked over time, and changes therein may be stored and timestamped. Therefore, a change in the stimulation program may be identified over time and may include a timestamp when the change occurs. The processor, such as the processor 57, may recall or evaluate the graph 100 to understand when a change in the stimulation program 158 has occurred. The processor 57 may then determine or evaluate the LFP trace 74 and identify a beginning and/or an end of an epoch. Therefore, a definition or evaluation of the LFP trace 74 to assist in identifying one or more epochs may be made by the processor 57 when evaluating the stimulation program 158 parameter change to assist in identifying one or more epochs of the LFP trace 74. Therefore, the processor system 57 may execute instructions to determine or identify any epoch of the LFP trace 74 based upon a recalling or evaluation of time stamps of the change in the stimulation program 158.

It is understood that any one or more of the parameters 130 may be used to assist in identifying one or more epochs of the LFP trace 74. Therefore, the rule change 168 defined in the change table 150 may be used either alone and/or in combination to assist in identifying an epoch of the LFP trace 74. Each of the parameters 130 may have a related rule change 168 included in the rule change table 150. The processor 57 by executing selected instructions may evaluate to the LFP trace 74, over time of each of the parameters 130, and the related rules changes in the rule change table 150 to assist in identifying one or more epochs of the LFP trace 74. The following discussion relating to each one or more possible parameters identify the respective parameter and a selected or possible rule change amount. It is understood, however, that the processor 57 may evaluate one or more of the parameters based upon the rule changes as discussed above and noted herein.

Another discrete stimulation parameter includes a stimulation electrode configuration 180, which may include a configuration of a lead or electrode thereon as an anode 182 or a cathode 184. The stimulation parameter of the configuration as a cathode 184 and configuration as an anode 182 may be included in the rule change chart 150. As discussed above the rule change may be identified in the column 168 and may include any change value 186 for the anode parameter 182 and any change in value 188 for the cathode parameter 184. As identified in the table 150 in the definition column 171, the configuration as the electrode as a cathode may be a negative electrode that may be delivering therapy and the configuration as an anode may be a positive electrode that is a reference or sink electrode. Therefore, changing the electrode configuration between or to either of the anode to configuration 182 for the cathode configuration 184 may be a rule change parameter identified in the rule change column 168. Identification or recalling that a change in value of the electrode to one or between either of the anode configuration 182 or the cathode configuration 184 may be recalled by the processor, such as the processor 57 to assist in identifying the beginning or end of an epoch of the LFP 74.

A further stimulation parameter may include a stimulation status 192. The stimulation status 192 may also include a rule change value 194 of any change. As defined in the table 150, the status may be whether stimulation is active (i.e., on) or not active (i.e., off). A further stimulation related parameter may include a stimulation mode 198 that includes any change as a value rule change 202. The stimulation mode may include whether the stimulation is based upon or changing relative to a current or voltage control, but may alternatively or additionally include cycling, adaptive (closed-loop), stimulation patterns, waveform shape, or combinations thereof. Further a stimulation related parameter may be a stimulation rate 206 and may include any change value 210 as a rule change parameter. The stimulation rate may include a frequency in stimulation change definition. A further discrete stimulation related parameter may be a stimulation pulse width 214. The pulse width 214 may also include an any change rule 218. The pulse width may relate to a stimulation duration including about 1 microsecond to about 1 second, including about 10 microseconds to about 1000 microseconds, including about 20 microseconds to about 450 microseconds, or any appropriate duration. Each stimulation duration is then interleaved with a no stimulation duration. Therefore, the pulse width 214 may be a duration of stimulation and any change in the duration of stimulation may be a change rule 168. An additional or alternative stimulation related parameter may be a stimulation limit parameter 217. The change rule for the stimulation limit parameter 217 may be based on the user-imposed limits and/or the limits of the system 219. Thus, the change rule 219 may relate to a change in the limits and/or the use of the limits of the stimulation system.

Additional discrete parameters may include those that are sense related 220. Sense related parameters may include a sense electrode configuration 224. The sense electrode configuration 224 may have a rule that any change 228 may define an epoch. The sense electrode configuration may include that an electrode is sensing brain activity, such as the LFP signal. The configuration of the electrode may include the position of the electrode, the contacts used to sense the signal, and the like. Another parameter that may be sense related 220 are one or more sensing thresholds 226. A sensing threshold or thresholds may refer to one or more LFP values against which the LFP signal may be compared. The addition or removal of one or more sensing threshold(s) may be used to define a change rule, or a change in the sensing threshold(s) itself(themselves) may be the change rule 227. The change rule may also be the result of a sensed signal or some measurement of the sensed signal relative to those one or more thresholds.

Further the discrete parameters may include a lead type 232. The lead type 232 may include any change as a rule change definition 236. The lead type may include a lead model, or other lead feature. For example, if a first lead is positioned within the subject 12 and removed and replaced with a different lead, the lead type may be determined to have changed.

Accordingly, a plurality of the system parameters may include discrete parameters. Each of the discrete parameters may include discrete changes, such as a stimulation program being on or off, a lead type being changed, or the like. Each of the discrete parameters may be used to define a rule that a change therein may be used to define an epoch change. Nevertheless, various parameters may include continuously monitoring or possibly changing parameters 142.

The continuous parameters may include stimulation related parameters 240. A stimulation related parameter 240 may include a stimulation amplitude 244. A stimulation amplitude 244 may have a rule change definition that refers to or determines an absolute or cumulative change that meets or exceeds a threshold as a change rule 248. In various embodiments, the cumulative change may be the direct average of the stimulation amplitude. In various embodiments, the cumulative change may be the summation of the consecutive variation in the stimulation amplitude, or any other computation of cumulative change. A threshold may be any appropriate absolute or proportional (percent) stimulation change amount. For example, changes by more than one volt or milliamp, or any other valid unit of stimulation, may define an absolute change threshold. The proportional or percent change may refer to the range (max value minus min value) of variation of the stimulation over a time window of interest. For example, a stimulation change greater than or equal to 10% of the range, greater than or equal to 20% of the range, or any other appropriate amount of proportional change may define a proportional change threshold. Additionally, a threshold may also refer to the stimulation limits, which may indicate the maximum range of stimulation values available to a user in a selected system. The stimulation limits may be used as the threshold that defines the change rule, or a change in the stimulation limits themselves may be the change rule. Therefore, a stimulation amplitude that meets or exceeds a threshold may be used as the change rule definition 248.

Further continuous parameters may include sense related parameters 252. Sense parameters 252 may include a sense frequency 256. The sense frequency 256 may include a rule change that is if a change is greater than or equal to a threshold of frequency change, a rule change determined 260. A sense frequency change threshold may be greater than one hertz (Hz), greater than three Hz, greater than five Hz, greater than 10 Hz, or any appropriate amount of change. Generally the sense frequency may be selected as any appropriate frequency, such as within a range of about 1 Hz to about 200 Hz, including about 12 Hz to about 30 Hz, and further including 16 Hz, 20 Hz, or any appropriate amount. Additionally, a selected sense frequency may have a range or band relative to that selected frequency. Therefore, a sense frequency of 60 Hz may sense a range of frequencies between about 58 Hz in about 62 Hz or any other appropriate range. Regardless of whether the sense frequency is a single frequency or a range or band of frequencies, changing the sense frequency by more than a selected amount, such as 5 Hz, may be a rule change parameter 260.

Change rules may also be general or relate to various parameters, which may be the same or in addition to those discussed above. For example, absolute changes of single parameters or ranges of parameters may be a change rule. Also, relative changes of single parameters or ranges of parameters may be a change rule. Additionally or alternatively, other measures of variation may be used as change rules for other continuous parameters.

In addition to each of the parameters, a selected number and selected ones of the parameters may be considered together. According to various embodiments, two or more of the parameters may be considered together which may also be referred to as a group or grouped. The group may be selected by a user, such as a physician/clinician. A group, for example, may include stimulation electrode configuration and stimulation pulse width. In addition, parameters may include state changes such as input states (e.g., taking medications), biomarker states (e.g., beta signal low or high), physiological states (e.g., body posture or movement), any other measured, detected, or reported patient, disease, or clinical state, any relevant time or time window, or any combination thereof.

In addition or alternatively to considering one or more selected parameters together, or two or more parameters grouped, one or more derivative parameters may be considered alone or grouped. A derivative parameter, for example, may be an individual parameter's rate or amount of change, measure of central tendency or spread, or any other computed or derived value based on one or more individual parameters or grouped parameters. The derivative parameter may be understood to be considered alone and/or together from the parameter or group of parameters from which it is derived.

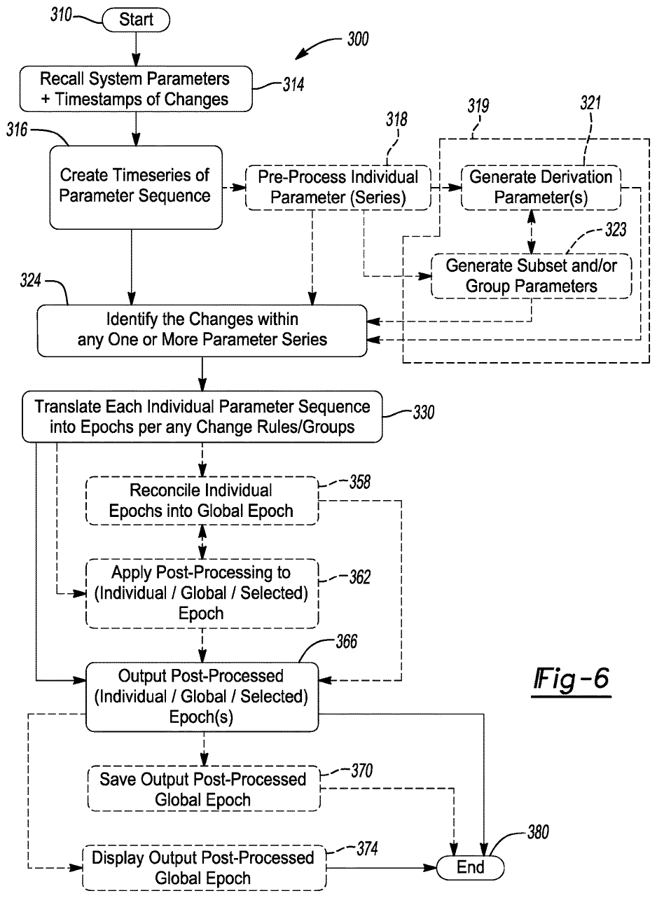

With continuing reference to FIG. 4 and FIG. 5, and additional reference to FIG. 6, a process 300 is illustrated for evaluating the system parameters 134 relative to the recorded LFP trace 74 and generating various epochs based thereon. Generally, any epoch, as referred to herein, may include a selected parameter and/or a combination of the various parameters that may be related to and/or is able to help define a timeframe or period of the LFP trace 74. This combination may be a unique combination and may include one or more of the parameters 130. It is understood that each of the parameters may be weighted with a selected and/or appropriate weight. For example, any one or more of the parameters may be weighted to have no weight to a weight such that a selected parameter may be the only parameter considered (e.g., weight of 1 or 100), or having any selected weight in evaluating or determining epoch of the LFP trace 74. In various embodiments, the weights may be appropriate weights and/or be limited to within a range. In various embodiments, the weights, if selected, may be analysis-driven coefficients that are determined by the user. The weight coefficients dictate whether the algorithm treats all the parameters equally or assigns different priority to specific target parameters.

Further, a reconciliation may include identifying the selected combination of parameters relative to or across hemispheres of the brain 28, over time, or any other appropriate parameter. The recorded parameters of the subject 12 may include parameters other than the recorded LFP identified or displayed in the LFP trace 74. The various parameters 130 may be used to assist in identifying epochs of the LFP trace 74. Thus, the reconciliation may include identifying which one of the parameters 130 may be used to identify epochs of the LFP trace and/or recording over time. Based on the parameters, epochs are defined that include one or a combination of the parameters to identify or segment the LFP trace over time into one or more individual epochs. A global epoch may then include a combination of the parameters 134 that may be displayed for evaluation by a user. For example, the parameters may include the stimulation status 192 and the stimulation mode 198 to identify epochs of the LFP trace 74. The global epochs may include a combination of these parameters relative to the LFP trace for evaluation by the user, as discussed further herein.

According to various embodiments, the process 300 may be used to identify epochs based upon parameters, which may include one or more of the parameters discussed above. The process 300 may also reconcile and generate a global epoch, in light of selected parameters, of the LFP traces 74. It is understood by one skilled in the art that discussion or epochs may refer to determining more than one epoch in a similar manner, but in a different time period. In particular, the various parameters may be used to identify time segments of the LFP trace into the epochs and the global epochs may include an illustration and/or comparison of a particular time period of the LFP trace 74 relative to another, particularly based upon the common parameters and/or different parameters for the LFP trace at those different times.

The process 300 may begin in start Block 310. After beginning the process 300 in start Block 310, system parameters and related timestamps or steps may be recalled or accessed in Block 314. The recalling of the system parameters may include recalling of the parameters 130 including the system parameters 134. The parameters 130 may be saved and/or recalled in an appropriate manner. For example, the parameters 130 and related information (e.g., time stamps of changes and/or values of the changes or type of changes) in the system parameters may be stored and/or recalled from a selected file, file type, or other data storage mechanism. If selected, the file may be stored in various portions, such as any appropriate memory portion including the memory in the IMD 16 as discussed above. The file may be accessed by the user system 54 and allow for collection and/or acquisition of system parameters and the time stamps of changes in the system parameters. As discussed above the change rule table 150 may include definitions of rules regarding the changes in the system parameters also identified in the system parameters as in the chart 130.

After accessing the file data in Block 314, a time sequence or series of a parameter sequence may be created or generated in Block 316. In creating or completing a time series or sequence in Block 316, a time series of each of the parameters and the time (e.g., timestamp) of their changed values may be created, thus serializing the parameter data. As illustrated in FIG. 2, the LFP traces 74 may also be recorded over time. Therefore, the created time series and parameter sequence in Block 316 may be overlaid or correlated to the time series or timeline of the LFP traces 74 as illustrated in the graph 70. Once the raw or un-serialized data is acquired in Block 314, it may be placed in a time series in Block 316 and compared to the LFP trace 74 in the graph 70. It is understood, however, that the LFP trace 74 need not be placed in a graph but may have various values also recorded over time and placed in a time series that is equivalent to or equal to the time series of the parameters created in Block 316.

The time series or serialized parameter data from block 316 may then be processed or pre-processed in Block 318. The parameter pre-processing may include various translations or transformations and processing of the data for various purposes. In various embodiments, a string of data may be processed into a single number, such as a string of readings over a period of time may be calculated into a single value (e.g., averaged over time). For example, a collection of data collected over a period of time, such as at intervals of less than a second, may be averaged or normalized into a value for each individual second. Further, the data may be made human readable, such as transforming or translating a wave into a numerical value.

Other translations or transformations may be made from the input data or the received data and may relate to different sensors or each of the sensors. As an example, if the stimulation is not applied for a period of time (such as a selected time frame threshold) an interpolation between stimulation values may be made and/or the data may be compressed. In various embodiments, for example, a stimulation may be programmed but data regarding a period of time may not have been received. Therefore, the pre-processing may include an interpolation of the data during selected time gaps. Also, a value may be applied to a clinically valid change, such as an LFP signal, duration of a seizure, or the like.

The pre-processing may be used to generate data for further processing in the process 300. Various optional processing portions may include an optional generation of parameters by derivation or grouping in a parameter generation sub-process or sub-processes 319. It is understood that the sub-processes 319 are optional and, therefore, are not required in the process 300.

Nevertheless, the optional pre-processing 319 may include a generation of a derivation parameter in block 321. The generated derivation parameter may include the derivation of one or more parameters and the discussion of a single parameter is only exemplary.

Generation of a derivative parameter may include deriving a new parameter from one or more parameters, as discussed above. For example, a new or derivative parameter may be a difference between two values measured in other parameters and/or differences in values measured in a single parameter, such as a change over time relative to initial state, change relative to a threshold, or difference relative to a threshold, or the like. Other parameters may include a selected value as a difference from an average or string of parameter values. Derivative values may also include a comparison of two parameters over time, a mean of two parameters over time, a comparison of two parameters at any selected time, or the like. Further, the derivative parameter may include a comparison between more than two parameters, such as more than two parameters of the parameters 130 discussed above or any other appropriate parameters. A sum of differences may also be used to define a new epoch, as discussed further herein.

A generation of new parameters may also include a grouping or determining a subset of parameters in optional block 323. A generation of a subset may include a selection of clinically relevant data collected from all possible sources and selected for the further processing in the process 300. Accordingly, the subset may include identifying one or more of the parameters 130 and selecting a subset of those parameters for further processing in the process 300. This may be useful if all of the collected data is not clinically relevant or appropriate for the process 300. In various embodiments, an example may include a sensing or stimulation where less than all contacts or electrodes of an implant may be received or recalled in block 314. In various embodiments, as understood by one skilled in the art, an implant may have a plurality of electrodes along a length thereof. However, a simulation with a selected number of electrodes, which is less than all of the electrodes, may be programmed for a system. Therefore, collecting data for all of the electrodes may not be clinically relevant and therefore a generation of a subset of the related parameter data may be made.

Additionally, or alternatively, parameters may be grouped. The grouping of parameters may include grouping of data received that may be otherwise separated, such as if data is collected in two columns. The two columns may then be put together into a single column, such as by addition or averaging. In an example, an electrode implant may include two electrodes or contacts that may receive data separately. This data may be combined, such as for the same instant of sensing or stimulation, into a single column of data. The grouping may also relate to how a physical electrode is implanted and/or program for stimulating a subject. For example, a selected polarity or fraction of stimulation may be grouped from a selected number of contacts or electrodes for further processing.

Therefore, the generation of derivative parameters, subset parameters, or grouping parameters may assist in defining or generating clinically relevant parameter data sets. The various parameters, may include those originally defined or recalled in block 314 and/or any that may be selected, derived, or grouped that may be used in the process 300 to assist in defining an epoch, as discussed herein.

After creating the time series in Block 316, or at any appropriate time, an identification of changes, including appropriate or selected changes, may be made in Block 324 to identify changes within any one or more parameter series. As discussed above and illustrated in the rule change table 150 each of the parameters may have changes that are identified relative to change rules 168. Once the parameter data is serialized in Block 316, the identification of changes, including all changes that meet the rules, may be made in Block 324. An identification of appropriate or selected changes, such as the rule change definition changes from column 168, may be made. The identified changes may be based on various parameters, parameter groups, derived parameters, etc., as discussed above, such as applying the detection rules to individual parameter changes in a sequence. Discussion herein of a single parameter change or change rule, however, is understood to relate to the appropriate type or parameter, such as individual, grouped, subset, etc.

Therefore, the serialized data from Block 316 may also be updated or tagged to include those changes that meet the rule change criteria in Block 324. A file, recalled file, or other data storage mechanism from block 314 may be annotated to include data regarding the parameter changes that meet the rule definitions from column 168. The parameter data may be identified and the changes thereof may be identified and included in data to assist in evaluating the LFP trace 74 relative to the parameter changes recalled in Block 314 and/or have been otherwise processed such as in the pre-processing 318 and/or the optional derived or grouped parameters in Block 319.

As discussed above, the identification of the parameter changes that meet the rule change 168 may be made in Block 324. In identifying all the changes and the parameters that meet the rule change, the processor may execute instructions to compare the parameter changes recalled in Block 314 to a lookup table, such as one that includes the inputs from the table 150. Thus, the system, including the use or system 54, may identify the various parameters and the respective changes that meets the rule change parameters from the table 150. This allows the output from Block 324 to include an identification of all of the parameter changes accessed from the data in Block 314.

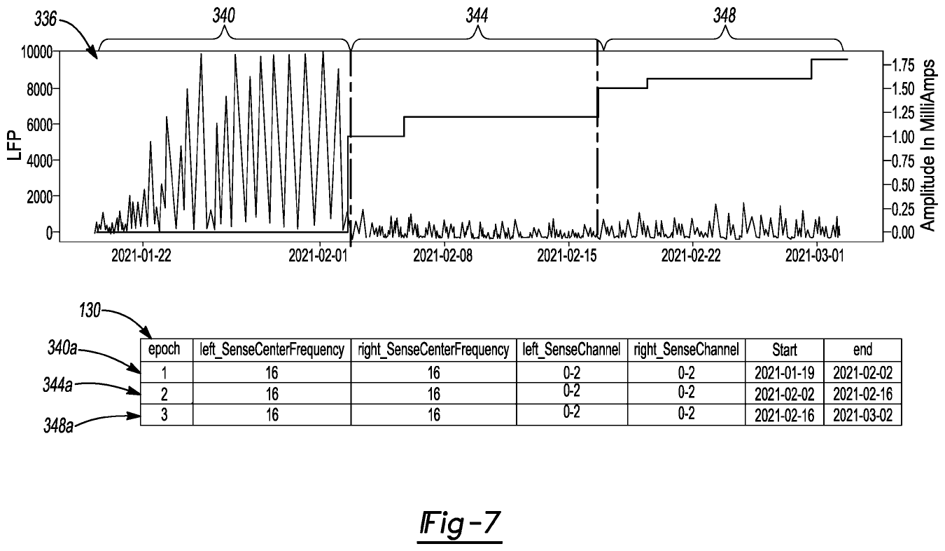

After identifying all of the changes in Block 324, a translation of any and/or all (including each) of the parameter sequences into epochs per the respective rule changes may be made in Block 330. In other words, the parameter sequences may be associated with the epochs and/or used to determine the epochs. As discussed above, the respective one or more parameters may be used to identify time (e.g., via timestamps and/or changes in the parameter over time) that relates to different epochs, such as based upon the changes of the parameters. With reference to FIG. 7, for example, a graph 336 illustrates the LFP trace 74 over time. The LFP trace may be broken into different epochs based upon various parameters, such as one or more of the parameters 130 exemplary illustrated in the first row of a table. As an example, each of the epochs may be identified based upon one or more of the parameters 130, as discussed above. The rule change definitions from table 150 may be applied to the parameters to identify the epochs. As exemplary illustrated in FIG. 7, the data may be identified in the table to include the rules of the parameters related to the epochs, the LFP graph 336 may also illustrate the various epochs, including a first epoch 340, a second epoch 344, and a third epoch 348. Each of the respective epochs relate to respective data or parameter changes as illustrated in the table in the respective rows 340a, 344a, and 348a. The initial identification of the epochs, including the translation of each individual parameter into epoch sequences in Block 330, therefore, may achieve the identification of various epochs, as illustrated in FIG. 7.

An optional reconciliation of each of the one or more epochs into a global epoch may be made in Block 358. According to the method 300, any processing or reconciliation of individual parameters or epochs into one or more global epochs is not required. A reconciliation into a global epoch may be performed to evaluate the inputs received into the process 300. The reconciliation may include an identification or translation of each of the epochs based upon each parameter that may be recorded relative to the LFP trace into a single epoch. For example, the system may include information regarding more than one parameter, such as including at least the parameters discussed above. For example, as illustrated in FIG. 7, a left and right sense frequency setting, a left and right sense channel change, or other appropriate parameter changes may be recalled. If only one parameter is measured the individual epoch may be set. If, however, many parameters are measured and used to identify any epoch, the individual epochs may be reconciled into a global epoch by way of, but not limited to, determining to what extent each parameter contributes to the formation of the global epoch. In one such example, the global epoch determination may include the correlation or weighting of a change in one or more than one parameter to identify the epoch. In the reconciliation of the global epoch on a subject-by-subject basis, different parameters and their respective changes may be weighted differently for different subjects where one parameter may be particularly relevant to an epoch relative to one subject.

Optional post processing may be applied in Block 362. The post processing of Block 362 is not required in the process 300, but may be useful as discussed herein. The post processing, if selected, may include one or more steps and may be performed on any one or more of the parameters or types, as discussed above, including to any or all selected individual epochs (i.e., Block 330) and/or the global epoch (i.e., Block 358). The post processing steps may include, but are not limited to, interpolating epoch durations that are shorter than a pre-determined minimum duration, or identifying which intervals pass a pre-determined minimum duration, or other appropriate post processing steps. The post-processing in block 362 may be applied to the epochs as determined above. In the process 300, an optional feedback path may also include post-processing in Block 362 the translated epochs from Block 330 and then reconciling them into a global epoch in Block 358. This would also allow further post-processing of the reconciled global epochs in Block 362.

Post-processing may include various features, such as applying a validity check based upon a duration of any epochs (e.g., each epoch meets a predetermined or selected minimum duration), a type of change or confirmation of a parameter change based upon one or more rules, or the like. Nevertheless, the post-processing of the epochs in Block 362 may allow for a confirmation or analysis of the translation of the epochs from Block 330. According to various embodiments, post-processing may include applying an epoch validity check (such as determining if each epoch meets minimum duration requirement), incorporating memory, filtering parameters of interest, interpolation of epoch gaps (i.e., if there is a missing data point in an epoch interpolate the missing data point), or unifying one or more epochs. Processing may also or alternatively include processing a signal to evaluate or determine a feature therein. For example, a signal and/or the parameter is a spectral feature of the electrical activity of the brain of the patient.

The process 300 may generate an output in Block 366. The output in Block 366 may include directly the translation of any and/or all (including each) of the parameter sequences into epochs per the respective rule changes made in Block 330. If the optional reconciliation to a global epoch in Block 358 and/or prost-processing in Block 362 is carried out, this may also or alternatively be the output in Block 366. The one or more epochs may be output in any appropriate manner in Block 366, such as to evaluate and/or assist in assessing response to a therapy for a subject, including treatment thereof. The process may then end in Block 380. The output in Block 366 may be the process 300 that is able to generate the one or more epochs as discussed above. Thus, the process 300 may be performed more than once for the same and/or different data to reach the output in Block 366.

The process 300, however, may also include various other processes or features for the output. For example, the one or more epochs may be optionally saved in a selected memory system, such as those discussed above, in Block 370. Saving the one or more epochs may allow for the user to recall the one or more epochs, such as for displaying with a display device, and/or for the making appropriate determinations of the treatment of the subject 12. Thus, the one or more epochs may also be displayed, optionally, in Block 374 such as on the display device or display screen 56. The one or more epochs may be displayed for use by the user, as discussed further herein.

Thus, also after saving in Block 370 and/or displaying in Block 374 the process 300 may end in Block 380. Ending the process 300 and Block 380 may be according to any appropriate step, such as after saving or storing the process one or more epochs and/or displaying it for appropriate purposes. The ending in Block 380 may also be determined by the user, such as when a selected number of epochs area determined and/or displayed.

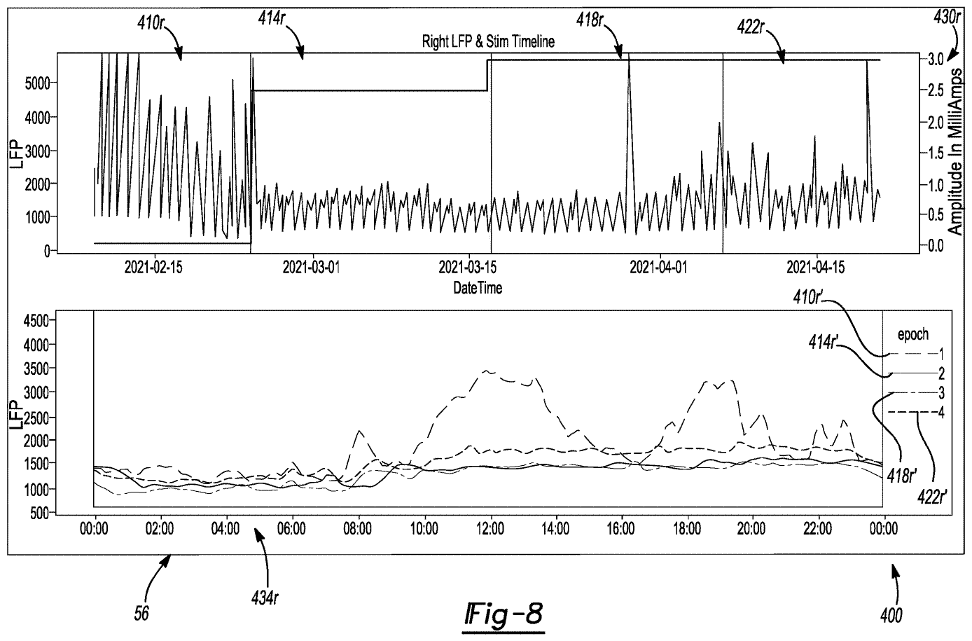

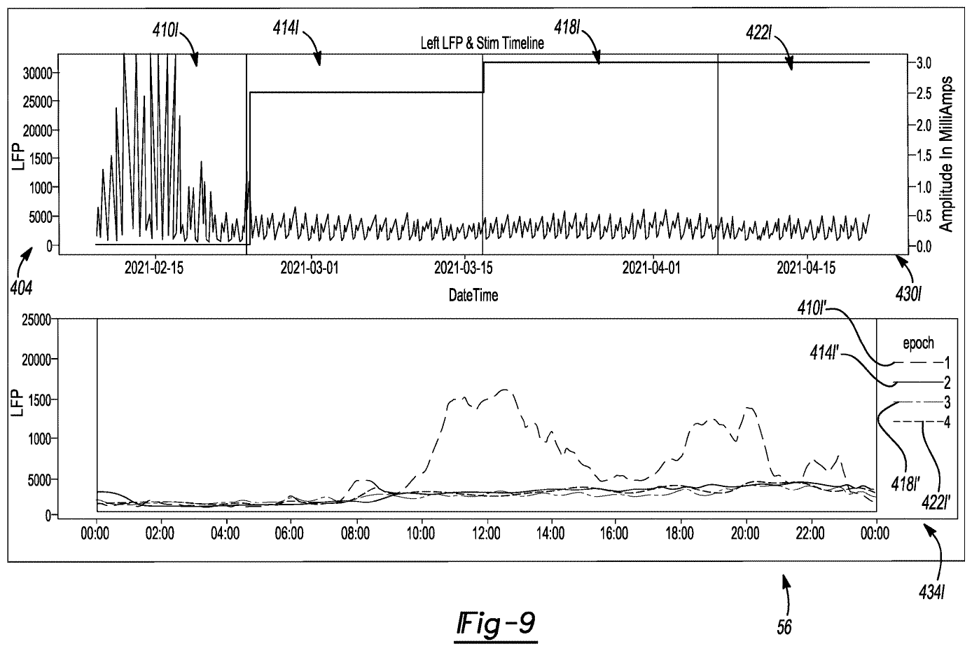

With continuing reference to FIGS. 1 through 7, and with additional reference to FIG. 8 and FIG. 9, the parameter data and/or the LFP trace data may be evaluated according to the process 300. The process 300 allows for the output and identification of each epoch. The identification of the epoch may then be used for various purposes, such as determining or evaluating an outcome metric of the subject. The outcome metric may relate and/or be used in a treatment and/or analysis of treatment of the subject 12. The outcome metric may be used by a clinician to assist in determining a value of a treatment (e.g., stimulation) and/or changing the treatment.

For example, as discussed above, treatment may include stimulation of and/or recording in one or both hemispheres of the brain 28 of the subject 12. FIG. 8 illustrates right hemisphere data 400 displayed on the display screen 56 and FIG. 9 illustrates left hemisphere data 404 illustrated on the display screens 56. It is understood that all the data may be displayed simultaneously and/or separately, as illustrated in FIGS. 8 and 9, or any other appropriate manner. The analysis of the process 300 may allow for the determination of the various epochs, as illustrated in FIGS. 8 and 9, and the comparison thereof, is illustrated in FIGS. 8 and 9. In various embodiments, the epoch determination and/or evaluation of the right and left hemispheres may be an example of global epochs (since all parameters for both hemispheres are considered when assigning these epochs and the epoch divisions are applied to both hemispheres).

For example, as illustrated in FIG. 8, a right hemisphere data may be separated into four epochs 410r, 414r, 418r, and 422r. As discussed above the epochs may relate to the parameters that are measured and recorded relative to the subject. Therefore, the epochs 410r, 414r, 418r, and 422r, may have respective and/or related left hemisphere epochs 410l, 414l, 418l, and 422l, as illustrated in FIG. 9. The epochs regarding the right and left hemispheres, therefore, may be the same based upon the parameters measured of the subject or recorded of the subject and may be illustrated relative to one another and/or separately as illustrated in FIGS. 8 and 9.

The respective data may include an LFP trace showing the individual epochs separated thereon in a first graph 430r related to the right hemisphere of the brain 28 and in a graph 430l related to the left hemisphere of the brain 28. An average of the LFP signal may be taken over each of the respective epochs and illustrated on a second graph 434r related to the right hemisphere and 434l related to the left hemisphere. The averaged LFP signal trace, illustrated in the graphs 434r, 434l may overlay the LFP traces over the same period of time as identified by the epochs. For example, the first LFP trace 410 may be displayed on the graph 434r, 434l as the respective traces 410r, 410l. Each of the other epochs (414, 418, 422) may be similarly illustrated to allow for the user to more readily compare respective LFP traces for the determined different epochs. Thus, the differentiation of the epochs may be made according to the process 300 and illustrated as shown in FIGS. 8 and 9 on the display 56.

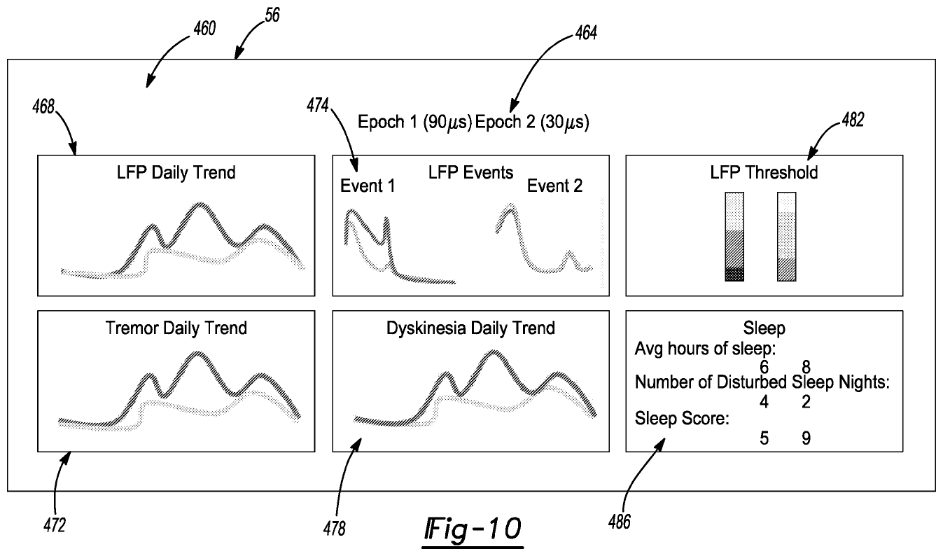

As discussed above, the process 300 may allow for the determination of an epoch of LFP traces based upon recorded parameters over time. Further, various subject outcome metrics may be determined and analyzed based on the determined epochs. As illustrated in FIG. 10, various information may be tabulated and displayed for the user on the display device 56 of the user system 54 in a user interface including a graphical user interface (GUI) 460, also referred to as a dashboard. According to various embodiments, for example, the dashboard 460 may illustrate information to the user. Information may include the epochs, such as determined by the process 300. The epochs may be illustrated such as with various color lines, dashed lines, etc. The acquired and analyzed data, including that discussed above, and any appropriate data may be displayed or illustrated, such as an LFP daily trend 468. The LFP daily trend 468 may illustrate a daily trend of LFP, such as over a 24 hour period, based upon the two different epochs.

In addition to the display of the determined epochs, such as the LFP daily trend 468, one or more subject outcome metrics may also be related to the determined epochs. In various embodiments, two epochs may be determined and displayed in the LFP daily trend 468. One or more subject outcome may also be correlated to the sequence of the determined epochs and displayed accordingly.

In various embodiments, the dashboard 460 may also illustrate a daily tremor trend 472 based upon measured tremors of the subject 12. As is understood by one skilled in the art, various sensor systems may be used to measure tremors, such as a wearable device and associated programs that may analyze and/or record the sensor data. One or more tremors may be sensed and time stamped and recorded. The tremor data may be separately recalled and/or may be recalled in the parameter data in Block 314. Given the time stamps of the measured tremors they may be related to determined epochs and displayed as the daily tremor trend 472. The occurrence of tremors may be a subject outcome metric that is measured and correlated to the epochs as illustrated in the daily tremor trend 472. The user may understand or select to illustrate a daily tremor trend based upon the identified epochs and/or use the tremor trend to identify the epochs.

One or more events correlated to the determined LFPs may also be illustrated in the dashboard 460. An event may be a patient triggered sensing feature. For example, a patient may trigger an event such as “Took Meds” and a sensor may capture a power spectral density plot when the patient triggers the event. Other events may include an identification of a subject's mood or emotional state, measurement or identification of being active or sedentary, or type of movement. As an example, in dashboard 460 two such events may be captured and correlated to the two epochs and may be displayed in the LFP trends 474. Further, the LFP trends 474 allow for a comparison of the two determined epochs illustrated in the dashboard 460. The power spectral density plots may be compared between different epochs (e.g., two or more) to evaluate if a change occurred.

The dashboard 460 may also include a dyskinesia daily trend 478. Various sensing systems may be used to identify dyskinesia and events thereof may be time stamped. The identification of dyskinesia and the timestamps thereof may be recalled for correlation to the determined epochs, such as recalled in Block 314. The occurrences of identified dyskinesia may be illustrated as correlated to the determined epochs in the dyskinesia daily trend 478. This may allow for an evaluation and determination of a subject outcome metric including dyskinesia trends correlated to the determined epochs in the LFP sequence.

The dashboard 460 may also identify or include information regarding LFP thresholds in LFP Thresholds 482. The LFP thresholds may relate or be defined by the user or a clinician. The LFP has a value and identified thresholds therein may be illustrated in the LFP Thresholds 482. The LFP Thresholds 482 may be illustrated as correlated to the determined epochs as well. As the epochs may be determined based on various or selected parameters and their respective changes, the threshold values and whether they are reached may be evaluated in the determined epochs, which relate to the changes in the parameters. Thus, the subject outcome metric regarding a value of the LFP and/or if it reaches a threshold may also be displayed and evaluated.

The dashboard 460 may also illustrate sleep patterns that may also be identified at 486. Sleep patterns may be measured and/or identified by the subject and/or an appropriate sensor, such as with a wearable device. The sleep patterns may include average hours of sleep, number of disturbed sleep nights, and a selected sleep score. Each of the measured or determined values may also be timestamped and correlated to the determined epochs. The sleep patterns related to the respective epochs may then be illustrated or displayed relative to the epoch times periods in portion 486.

Accordingly, the dashboard 460 may be used to display various recorded data of the subject 12 based upon the determined epochs. The epochs may be determined based upon the system parameters 130 and the process 300. Various subject outcome metrics may also be timestamped and then correlated to the determined epochs. The dashboard 460 may display the epochs and/or some feature or metric relative to the epoch. In the dashboard 460 one or more of the selected parameters, a subject's response, a subject's symptom, and/or other measured feature may also be displayed correlated or relative to the identified epochs.

According to various processes disclosed herein one or more outputs may be made. According to various embodiments a selected system, which may include a processor module, may be programmed to execute instructions to carry out the processes disclosed herein and generate selected outputs.

Outputs may include various subject outcome metrics that may be associated with the various epochs, such as the tremor trend data disclosed herein. Thus, the subject outcome metric may be determined for one or more epochs and the subject outcome metric may be associated with parameters and/or the determined epoch.

Further, the output may include an outcome metric that associates one or more parameters across the determined epochs.

As discussed above the epochs may relate to a time period. The information displayed on the dashboard 460 may also be measured over time. The information may include one or more of a subject's response, a subject's symptom, a subject's biophysical state, or other appropriate subject outcome metric. The subject outcome metrics may be outcomes, measured or subjective, of the subject that relate to desired or undesired outcomes in light of treatment, such as tremor patterns or sleep information. The timestamp of the tremor patterns and/or sleep information may be correlated to the epochs and displayed relative thereto in the dashboard 460.

A subject's response may include a change in LFP such as determined by the LFP thresholds, including an elevation and/or suppression of the LFP signal. The subject's response may include sleep state which may further related to a circadian rhythm. The subject's response may include a number of symptom events that are identified and timestamped. The subject's response may further include a tremor and/or dyskinesia score. A subject's heart rate variability and/or the hearts specific rate at a given time may be included as a subject's response. Thus, the subject's response may include one or more responses of the subject to a change in one or more of the parameters.

A subject's symptom may include a rigidity of the subject at a determined time stamp. The subject's symptom may include a tremor or dyskinesia score or state at a given timestamp. Further, a subject's symptom may include any measured or selected motor disorder symptoms.

A subject's biophysical state may include whether the subject currently is taking a medication and/or has an effective concentration of a medication as related to time. The subject's biophysical state may include a determination of a time stamp of an active or sedentary state and/or type or amount of movement. The subject's biophysical state may include a timestamp of the subject being awake or asleep. Also, the subject's biophysical state may include a timestamped identification of the subject's mood and/or emotional state.

Therefore, a user may identify or select to have various parameters evaluated in the process 300 or outcomes to be identified and displayed each box of the dashboard 460. The epochs may be used to evaluate the subject and the related LFP trends or LFP recordings. Thus, the user may display or view selected epochs and evaluate treatment of the subject, alter treatment of the subject, determine disease progression and/or success of therapy, or other appropriate information.

### EXAMPLES

### Example 1

A system to determine an outcome metric of a subject undergoing a therapy, comprising: a processor module configured to: at least receive signal data regarding a signal from the subject with one or more corresponding signal time stamps; at least receive data regarding one or more parameters with one or more corresponding parameter time stamps; execute instructions to: identify one or more changes in the parameters based on one or more change rules; determine one or more epochs based on the identified one or more changes and the parameter time stamps that correspond to the identified one or more changes; output at least one first outcome metric for the determined epochs, wherein the first outcome metric comprises associating the signal data within at least one determined epoch.

### Example 2

The system of Example 1, wherein the processor further executes instructions to output a second outcome metric, wherein the second outcome metric comprises associating signal data to one or more parameters across the determined epochs.

### Example 3

The system of Example 1, wherein the first outcome metric further comprises associating the signal data to one or more parameters within the determined epochs.

### Example 4

The system of Example 1, wherein the first outcome metric includes a visual display of the signal data compared to the one or more parameters within a single determined epoch.

### Example 5

The system of Example 2, wherein the second outcome metric includes a visual display of signal data compared to the one or more parameters across two or more epochs.

### Example 6

The system of Example 2, wherein the first outcome metric, second outcome metric, or both correspond to the subject's response to the therapy; wherein the therapy includes a stimulation therapy.

### Example 7

The system of Example 2, wherein the first outcome metric, second outcome metric or both correspond to a symptom associated with a neurological disorder.

### Example 8

The system of Example 1, wherein the first outcome metric, second outcome metric or both correspond to a biophysical state of the patient.

### Example 9

The system of Example 1, wherein the signal is a local field potential.

### Example 10

The system of Example 1, wherein determine one or more epochs based on the identified one or more changes and the parameter time stamps that correspond to the identified one or more changes includes determining a selected number of epochs at least by reconciling all of the identified changes and the parameter time stamps that correspond to the identified changes.

### Example 11

The system of Example 1, wherein the processor module is configured to execute further instructions to output the determined one or more epochs and generate a visual display of the determined one or more epochs.

### Example 12

The system of Example 1, wherein the one or more parameters includes at least one of a stimulation program, a stimulation electrode configuration as a cathode, a stimulation electrode configuration as an anode, a stimulation pulse width, a stimulation status, a stimulation mode, a stimulation rate, a stimulation amplitude, a lead type, a sense electrode configuration, or a sense frequency.

### Example 13

A method for determining an outcome metric of a subject undergoing a therapy, comprising: receiving signal data regarding a signal from the subject with one or more corresponding signal time stamps; receiving data regarding one or more parameters with one or more corresponding parameter time stamps; identifying one or more changes in the parameters based on one or more change rules; determining one or more epochs based on the identified one or more changes and the parameter time stamps that correspond to the identified one or more changes; and outputting at least one first outcome metric for the determined epochs, wherein the first outcome metric comprises associating the signal data within at least one determined epoch.

### Example 14

The method of Example 13, further comprising: outputting a second outcome metric, wherein the second outcome metric comprises associating signal data to one or more parameters across the determined epochs.

Example 15

The method of Example 13, wherein outputting the at least one first outcome metric further comprises associating the signal data to one or more parameters within the determined epochs.

Example 16

The method of Example 13, further comprising: generating a visual display of the signal data compared to the one or more parameters within a single determined epoch as outputting the first outcome metric.

Example 17

The method of Example 14, further comprising: generating a visual display of signal data compared to the one or more parameters across two or more epochs as outputting the second outcome metric.

Example 18

The method of Example 14, wherein the first outcome metric, second outcome metric, or both correspond to the subject's response to the therapy; wherein the therapy includes a stimulation therapy.

Example 19

The method of Example 14, wherein the first outcome metric, second outcome metric or both correspond to a symptom associated with a neurological disorder.

Example 20

The method of Example 13, wherein the first outcome metric, second outcome metric or both correspond to a biophysical state of the patient.

Example 21

The method of Example 13, wherein the signal is a local field potential.

Example 22

The method of Example 13, determining the one or more epochs based on the identified one or more changes and the parameter time stamps that correspond to the identified one or more changes includes determining a selected number of epochs at least by reconciling all of the identified changes and the parameter time stamps that correspond to the identified changes.

Example 23

The method of Example 13, further comprising: outputting the determined one or more epochs; and generating a visual display of the determined one or more epochs.

Example 24

The method of Example 13, wherein receiving data regarding one or more parameters includes receiving data regarding at least one of a stimulation program, a stimulation electrode configuration as a cathode, a stimulation electrode configuration as an anode, a stimulation pulse width, a stimulation status, a stimulation mode, a stimulation rate, a stimulation amplitude, a lead type, a sense electrode configuration, or a sense frequency.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

1.-15. (canceled)

16. A system for evaluating stimulation therapies delivered by a medical device implanted in a patient's brain, the system comprising:
one or more processors; and
a memory operably coupled to the one or more processors and storing instructions that, when executed by the one or more processors, cause the system to perform operations comprising:
receiving, from the medical device, signal data characterizing a bioelectrical signal from the patient's brain;
receiving parameter data characterizing one or more parameters used to stimulate the patient's brain;
determining one or more parameter changes in the parameter data;
determining, based on the one or more parameter changes, one or more epochs in the signal data; and
associating an outcome metric with at least one of the one or more determined epochs.

17. The system of claim 16, wherein the outcome metric corresponds to a symptom associated with a neurological disorder of the patient.

18. The system of claim 17, wherein the symptom is a tremor.

19. The system of claim 16, wherein the outcome metric comprises a disease state biomarker based on the signal data.

20. The system of claim 16, wherein the operations further comprise communicating the outcome metric to a clinician or user.

21. The system of claim 20, wherein communicating the outcome metric to a clinician or user comprises displaying the outcome metric on a display.

22. The system of claim 16, wherein the one or more parameter changes comprises one or more changes to the following parameters: stimulation program, stimulation electrode configuration, stimulation pulse width, stimulation status, stimulation mode, stimulation rate, stimulation amplitude, lead type, sense electrode configuration, sense frequency, sensing threshold, or stimulation limits.

23. The system of claim 16, wherein determining one or more parameter changes in the parameter data comprises:
identifying a first parameter value for a parameter in the parameter data at a first timestamp,
identifying a second parameter value for the parameter in the parameter data at a second timestamp different than the first timestamp, and
determining whether a difference between the first parameter value and the second parameter value exceeds a predetermined threshold.

24. The system of claim 16, wherein the one or more parameter changes comprises a discrete parameter change including a type change, a status change, or a step change.

25. The system of claim 16, wherein the one or more parameter changes comprises a first parameter change and a second parameter change, and wherein determining the one or more epochs in the bioelectrical data comprises determining whether the first parameter change and the second parameter change correspond to the same time period.

26. A method for evaluating stimulation therapies delivered by a medical device implanted in a patient's brain, the method comprising:
receiving, from the medical device, signal data characterizing a bioelectrical signal from the patient's brain;
receiving parameter data characterizing one or more parameters used to stimulate the patient's brain;
determining one or more parameter changes in the parameter data;
determining, based on the one or more parameter changes, one or more epochs in the signal data; and
associating an outcome metric with at least one of the one or more determined epochs.

27. The method of claim 26, wherein the outcome metric corresponds to a symptom associated with a neurological disorder of the patient.

28. The method of claim 27, wherein the symptom is a tremor.

29. The method of claim 26, wherein the outcome metric comprises a disease state biomarker based on the signal data.

30. The method of claim 26, further comprising communicating the outcome metric to a clinician or user.

31. The method of claim 30, wherein communicating the outcome metric to a clinician or user comprises displaying the outcome metric on a display.

32. The method of claim 26, wherein the one or more parameter changes comprises one or more changes to the following parameters: stimulation program, stimulation electrode configuration, stimulation pulse width, stimulation status, stimulation mode, stimulation rate, stimulation amplitude, lead type, sense electrode configuration, sense frequency, sensing threshold, or stimulation limits.

33. The method of claim 26, wherein determining one or more parameter changes in the parameter data comprises:
identifying a first parameter value for a parameter in the parameter data at a first timestamp,
identifying a second parameter value for the parameter in the parameter data at a second timestamp different than the first timestamp, and
determining whether a difference between the first parameter value and the second parameter value exceeds a predetermined threshold.

34. The method of claim 26, wherein the one or more parameter changes comprises a discrete parameter change including a type change, a status change, or a step change.

35. The method of claim 26, wherein the one or more parameter changes comprises a first parameter change and a second parameter change, and wherein determining the one or more epochs in the bioelectrical data comprises determining whether the first parameter change and the second parameter change correspond to the same time period.
